(19) United States
(12) Patent Application Publication
(10) Pub. No.: US 2014/0230819 A1
(43) Pub. Date: Aug. 21, 2014

(54) SYSTEMS AND METHODS FOR MONITORING PATIENTS FOR RISK OF VENTILATOR ASSOCIATED EVENTS

(71) Applicant: COVIDIEN LP, Mansfield, MA (US)

(72) Inventors: Robert Rawlins, Westminster, CO (US); Robert T. Boyer, Longmont, CO (US); Gary Milne, Louisville, CO (US); Gauri Joglekar, Fort Collins, CO (US)

(73) Assignee: COVIDIEN LP, Mansfield, MA (US)

(21) Appl. No.: 13/768,769

(22) Filed: Feb. 15, 2013

(51) Int. Cl. A61M 16/00; A61M 16/04; A61M 16/06; A61M 16/08

(52) U.S. Cl. CPC A61M 16/0057; A61M 16/0875; A61M 16/04; A61M 16/06
USPC 128/204.23

(57) ABSTRACT

A system for monitoring a patient on ventilation includes a ventilator system configured to provide mechanical ventilation in accordance with a FiO₂ setting and a PEEP setting, a temperature sensor configured to sense body temperature, one or more input devices configured to receive patient information, e.g., WBC count information, an information processing system, and a display device. The information processing system is communicatively coupled to the ventilator system, temperature sensor, and input device, and is configured to receive, process, and output FiO₂ information, PEEP information, temperature information, and WBC count information. The display device is configured to display the information output from the information processing system graphically as a function of time. The system may further be configured to provide notifications and alerts and/or to generate and transmit reports based on the information received by the information processing system. Methods for monitoring a patient on ventilation are also provided.

### TECHNICAL FIELD

The present disclosure relates to patient monitoring and, more particularly, to systems and methods for monitoring patients for risk of Ventilator Associated Events (VAEs).

### BACKGROUND

It has been estimated that more than 300,000 patients receive mechanical ventilation in the United States each year. Mechanical ventilation is often an essential, life-saving therapy for critically ill patients and patients experiencing respiratory failure. However, despite the obvious benefits, patients on mechanical ventilation are at an increased risk for complications such as Ventilator-Associated Pneumonia (VAP), sepsis, Acute Respiratory Distress Syndrome (ARDS), pulmonary embolism, barotraumas, and pulmonary edema. These complications may necessitate extended ventilation use, longer hospital stays, increased costs, and/or may increase the risk of disability or death.

Recently, the Centers for Disease Control and Prevention (CDC) has established a protocol for the surveillance and reporting of so-called Ventilator Associated Events (VAEs). This Ventilator Associated Event Protocol is designed to facilitate the gathering of data related to known occurrences of VAEs so that the effectiveness of prevention strategies can be assessed.

### SUMMARY

The present disclosure relates to systems and methods for monitoring a patient on ventilation. Systems provided in accordance with the present disclosure include, for example, a ventilator system, a temperature sensor, one or more input devices, an information processing system, and a display device. The ventilator system is configured to provide mechanical ventilation in accordance with a FiO₂ setting and a PEEP setting. The temperature sensor is configured to sense body temperature. The one or more input devices are configured to receive patient information, e.g., WBC count information. The information processing system is communicatively coupled to the ventilator system, temperature sensor, and input device(s) and is configured to receive, process, and output FiO₂ setting information, PEEP setting information, body temperature information, and WBC count information. The display device is configured to display the FiO₂ setting information, PEEP setting information, body temperature information, and WBC count information output from the information processing system graphically as a function of time. The system may further be configured to receive at least some of the above-information from another system, e.g., a patient's Electronic Medical Record (EMR), an Admission, Discharge, and Transfer (ADT) electronic file, lab data, etc., provide notifications and alerts and/or to generate and transmit reports, e.g., to another system, based on the information received by the information processing system. Methods for monitoring a patient on ventilation are also provided.

The aspects and features of the present disclosure are advantageous in that they provide a user with real-time information related to parameters, conditions, and/or sensed data of a patient on mechanical ventilation and, more particularly, real-time information indicating a patient's risk of experiencing a Ventilator Associated Event (VAE). The aspects and features of the present disclosure are also advantageous in that the real-time information may be presented as visual graphical representations indicating a patient's level of risk, e.g., using color-coding, in accordance with the parameters, conditions, and/or sensed data, thus presenting the user with trend information regarding the patient's risk. The aspects and features of the present disclosure are further advantageous in that automatic notifications and/or automatically generated reports may be provided in real-time based on the current information and/or trending information related to the parameters, conditions, and/or sensed data, either individually or collectively.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic illustration of a patient care and monitoring system provided in accordance with the present disclosure;

FIG. 2 is a schematic illustration of one hardware and software configuration for use with the system of FIG. 1;

FIG. 3 illustrates a display screen provided in accordance with the present disclosure, as presented to a user;

FIG. 4 illustrates another display screen provided in accordance with the present disclosure, as presented to a user; and

FIG. 5 is a flow diagram illustrating one implementation of the present disclosure.

### DETAILED DESCRIPTION

Provided in accordance with the present disclosure are systems and methods for monitoring a patient on a mechanical ventilator for risks of Ventilator Associated Events (VAEs) and for observing the occurrence of VAEs. VAEs generally fall into one of three categories: Ventilator Associated Conditions (VACs), Infection-related Ventilator Associated Complications (IVACs), and possible or probable Ventilator Associated Pneumonia (VAP). Certain parameters, conditions, and/or sensed data, alone or in combination with other parameters, conditions, and/or sensed data, have been found to indicate an increased risk that a patient may suffer a VAE, that a VAE is ongoing, and/or that a VAE has occurred. As such, past information, current information, and/or trending information regarding these parameters, conditions, and/or sensed data can be utilized to determine a patient's risk of suffering a VAE and, in some instances, which type (or types) of VAE presents an increased risk to the patient, as well as for gathering data related to an occurring or previously-occurred VAE. As will be described in detail below, the systems and methods of the present disclosure monitor, compile, and output, e.g., via visually displaying, providing notifications (audible, text, etc.), generating reports, etc., past, current, and trending information regarding these parameters, conditions, and sensed data, thus facilitating a user in readily and early ascertaining a patient's current status and risk of suffering a VAE, whether the patient is currently experiencing a VAE, and/or whether the patient has experienced a VAE.

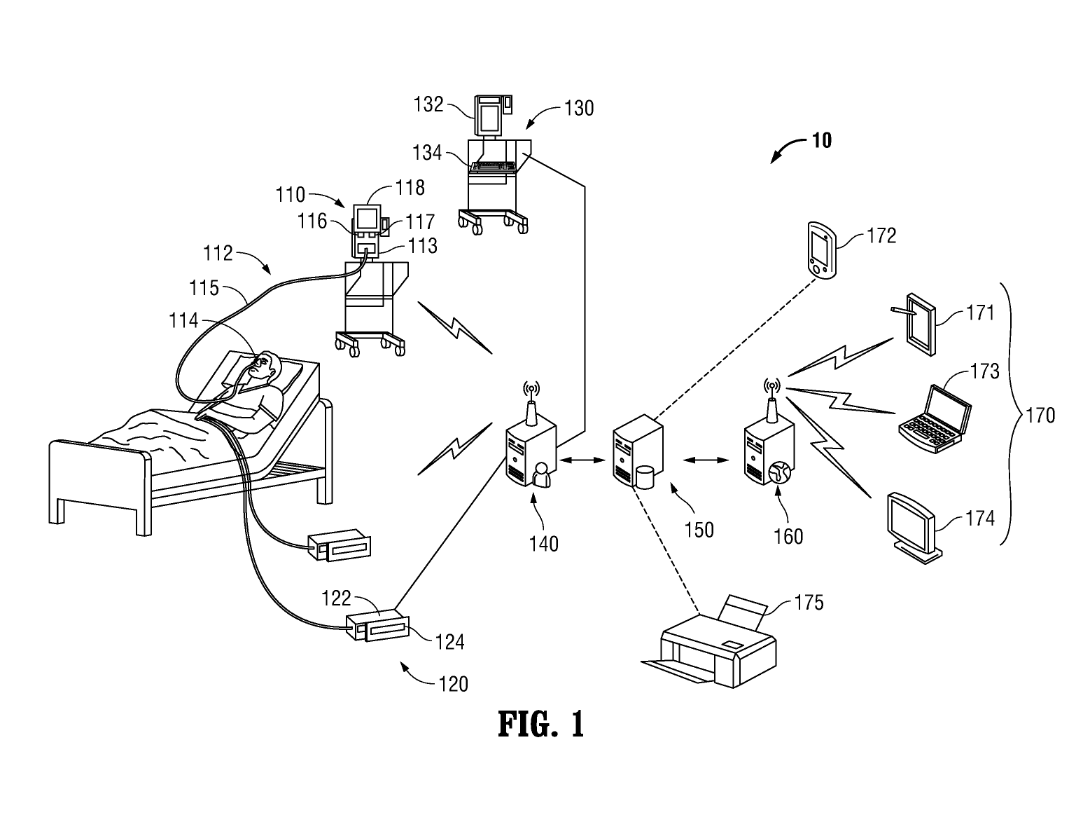

Referring to FIG. 1, an exemplary system provided in accordance with the present disclosure is shown generally identified by reference numeral 10. System 10 includes one or more patient care devices 110, one or more patient monitoring devices 120, one or more bedside devices 130, one or more data servers 140, one or more application servers 150, one or more web servers 160, and one or more remote devices 170. For the purposes herein, exemplary system 10 is generally described, although the aspects and features of the present disclosure may be implemented, incorporated, or utilized with any other devices, systems, and combinations thereof.

The one or more patient care devices 110 may include, for example, a ventilator system 112. Ventilator system 112 may be any suitable ventilator system, e.g., the Puritan Bennett™ 840 Ventilator System sold by Covidien LP of Boulder, Colo., USA, and generally includes a bedside unit 113, a patient interface member 114, e.g., an insertion tube (for invasive ventilation) or mask (for noninvasive ventilation), and a pair of tubes 115, e.g., an inflow tube and an outflow tube, interconnecting the bedside unit 113 and patient interface member 114. Bedside unit 113 controls the supply of oxygenated air to the patient and includes at least a first input 116 for allowing a user to set a desired Fraction of inspired Oxygen (FiO₂) level and a second input 117 for allowing a user to set a desired Positive-End Expiratory Pressure (PEEP). Bedside unit 113 may further include a display 118 for displaying relevant data relating to ventilation system 112 and/or the patient. Bedside unit 113 may be coupled to one or more servers 140, 150, 160, e.g., data server 140, either wirelessly or via a wired connection. Bedside unit 113 may include any suitable software, firmware, and hardware for the above purposes.

The one or more patient monitoring devices 120 may include, for example, a temperature sensor 122 for measuring a patient's body temperature and/or any other suitable device(s) for visual monitoring, audible monitoring, monitoring of physical characteristics, physiological conditions, and/or other measurables. Temperature sensor 122 (and/or the other patient monitoring devices 120) may be configured to display temperature data (or other data) on a visual display 124 thereof and/or may be configured to relay temperature data (or other data) to one or more servers 140, 150, 160, e.g., data server 140. Temperature sensor 122 may be wirelessly coupled to data server 140, or may be coupled to data server 140 via a wired connection. Temperature sensor 122 may include any suitable software, firmware, and hardware for the above purposes.

Bedside device 130 may include a display 132 and a user input 134, e.g., a touch-screen display or keyboard and mouse. Bedside device 130 is employed to display relevant parameters, conditions, sensed data, and/or other patient information on display 132 at a patient's bedside. Bedside device 130 is further configured to receive user input data via user input 134 for display on display 132 and/or for transmission to data server 140 (and/or the other servers 150, 160). Exemplary information input into bedside device 130 may include medical care information, e.g., the patient's drug administration schedule, other measured data, e.g., the patient's white blood cell (WBC) count, and/or biographical or other observed data/notes. Bedside device 130 may be wirelessly coupled to data server 140, or may be coupled to data server 140 via a wired connection. Bedside device 130 may include any suitable software, firmware, and hardware for the above purposes. Alternatively or additionally to bedside devices 130, patient monitoring devices 120, and patient care devices 110, information relating to the patient may be automatically pulled from another system, e.g., a patient's Electronic Medical Record (EMR), an Admission, Discharge, and Transfer (ADT) electronic file, lab data, etc.

Data server 140, application server 150, and web server 160 are coupled to one another and between remote devices 170 and the local devices, e.g., ventilator system 112, temperature sensor 122, and bedside device 130, for storing, processing, and/or transmitting information therebetween. More specifically, one or more of servers 140, 150, 160, e.g., data server 140, are configured to store information, e.g., the parameters, conditions, sensed data, reports, and/or other information, in a database and to process the information. Servers 140, 150, 160 are further configured to cooperate with one another to transmit information between the servers 140, 150, 160, other systems, e.g., a patient's EMR, an ADT electronic file, lab data, etc., ventilator system 112, temperature sensor 122, bedside device 130, and/or remote devices 170. Servers 140, 150, 160 may include any suitable software, firmware, and hardware for these purposes and may establish the above-described communication via wired and/or wireless communication.

Remote devices 170 request and receive information, e.g., the parameters, conditions, sensed data, and/or other information, process the information, if needed, and display the information to a user, e.g., via a display monitor, user interface, browser, and/or application running on the remote device 170, or otherwise output the information to a user, e.g., print a generated report containing the information. Remote devices 170 may further be configured to receive input from a user, e.g., to input information, control display or output of the information, set parameters, reset notifications, etc. Information input into remote devices 170 may include medical care information, e.g., the patient's drug administration schedule, other measured data, e.g., the patient's white blood cell (WBC) count, and/or biographical or observed data. Remote devices 170 may include one or more tablet PCs 171, smartphones 172, laptop computers 173, display monitors 174, printers 175, or other suitable devices and may incorporate any suitable software, firmware, and hardware for the above purposes.

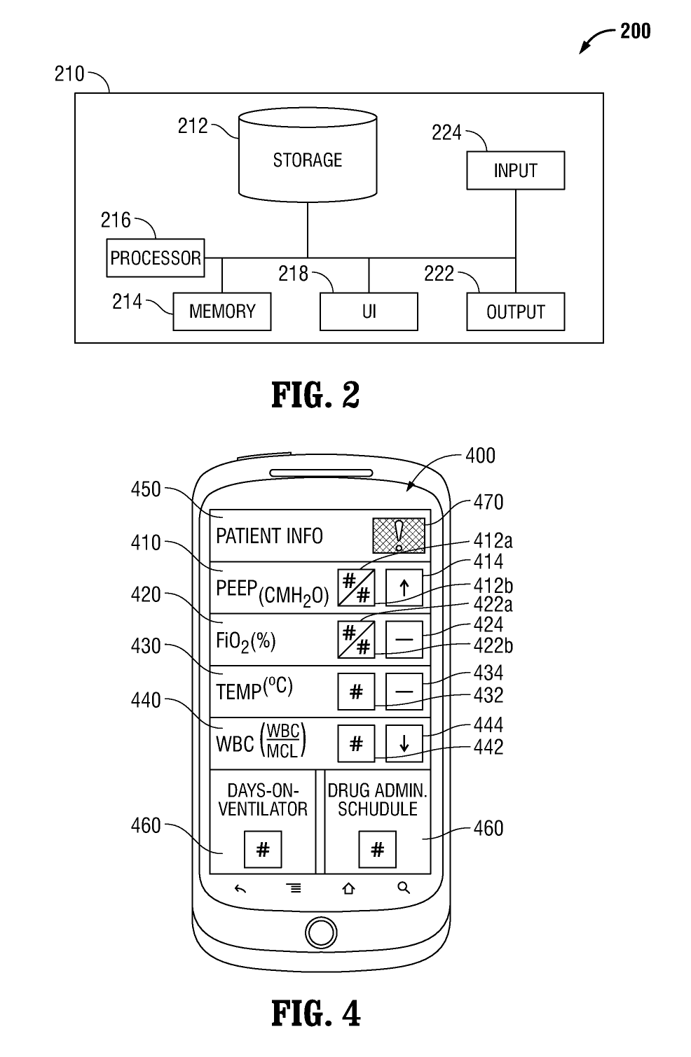

Turning now to FIG. 2, in conjunction with FIG. 1, one configuration of hardware and software components for receiving/transmitting information, e.g., the parameters, conditions, sensed data, and/or other information, processing the information, receiving user input, and/or displaying the information or otherwise outputting the information in accordance with the present disclosure is shown generally identified by reference numeral 200. Configuration 200 may be embodied within one or more of patient care devices 110, patient monitoring devices 120, bedside devices 130, servers 140, 150, 160, and remote devices 170, or may be implemented across one or more of patient care devices 110, patient monitoring devices 120, bedside devices 130, servers 140, 150, 160, and remote devices 170. That is, receiving/transmitting the information and user input, processing the information, and outputting the information for display or other output may be performed locally, e.g., at one of patient care devices 110, patient monitoring devices 120, bedside devices 130, on one or more servers 140, 150, 160 for distribution to the various devices 110, 120, 130, 170, e.g., across a network, at the remote devices 170 themselves, or in any combination of the above. For the purposes of simplicity, configuration 200 will be described herein as embodied in a system 210, keeping in mind that system 210 may be incorporated into or across any or all of the components of system 10.

System 210 generally includes a storage 212, a memory 214, a processor 216, a user interface (UI) 218, an output 222, and an input 224. Storage device 212 may include any suitable component(s) operable for storing information received via input 224, such as, for example, a magnetic disk, flash memory, optical disk, or other suitable data storage device. Memory 214 may include any computer memory, e.g., RAM or ROM, mass storage media, removable storage media, combinations thereof, or any other suitable computer-readable storage medium, storing instructions for causing processor 216 to execute particular functions, e.g., to process the information. Processor 216 may include any suitable component(s), e.g., a central processing unit (CPU), operable to execute instructions stored in memory 214 to process and manipulate information, e.g., stored in storage device 212 or received via input 224, for output to UI 218 or output 222. Processor 216 is further configured to receive, via input 224 and/or UI 218, information, data, and/or control parameters for processing and manipulating the information in accordance with user-selected settings and user input. UI 218 functions to output the processed information for visual display, e.g., in graphical and/or numerical form, to the user and/or allows for the input of information, data, setting of parameters, etc., by the user. Output and input 222, 224, respectively, are provided to facilitate communication between system 210 and the other components of system 10. In particular, input 224 is configured to receive information to be processed, e.g., data from ventilator system 112, temperature sensor 122, and bedside device 130 (or other device where user-input data is provided).

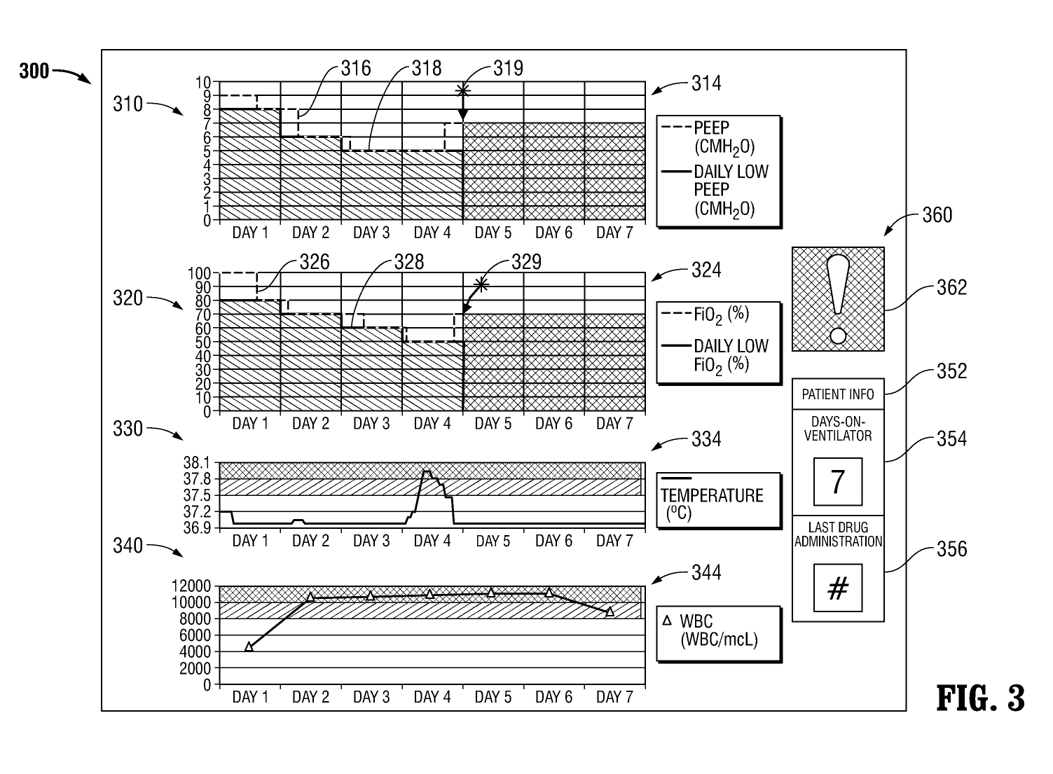

Turning now to FIG. 3, in conjunction with FIGS. 1 and 2, a main display screen 300 is show displaying exemplary information as output by UI 218. Main display screen 300 may represent display 118 of ventilator system 112 (or other patient care device 110), display 124 of temperature sensor 122 (or other patient monitoring device 120), display 132 of bedside device 130, or a monitor, display, etc. of any of remote devices 170. That is, depending on the configuration of system 10, the user may view display screen 300 on one of the patient care devices 110, patient monitoring devices 120, bedside devices 130, remote devices 170, or any other suitable device.

Display screen 300 includes first, second, third, and fourth graph image areas 310, 320, 330, 340 for graphically displaying current and trending information relating to PEEP, FiO₂, temperature, and WBC count, respectively. The orientation, ordering, and/or relative positioning of graph image areas 310, 320, 330, 340 may be selected and/or changed in accordance with user preference. Patient information, e.g., biographical information, drug administration schedule information and/or days-on-ventilator information, is also displayed on display screen 300 via text boxes 352, 354, 356. Display screen 300 further includes a status indication area 360 for displaying a status indicator 362 based upon current and/or trending information, e.g., PEEP, FiO₂, temperature, WBC count, and other patient information. Each of the above-noted features of display screen 300 will be described in detail, in turn, below.

First graph image area 310 is configured for displaying a PEEP graph 314 representing the PEEP level (measured in cmH₂O) set in ventilator system 112 as a function of time, e.g., for the past 7 days (although other timeframes are also contemplated). PEEP graph 314 includes an actual PEEP indicator line 316 (the broken line) and a daily low PEEP indicator line 318 (the solid line) superimposed on one another. The area under the daily low PEEP indicator line 318 is shaded or colored in accordance with a coding protocol, which will be described below. Further, alert indicators 319 may be provided in graph 314 at particular points of concern, as will also be described below. As can be appreciated, PEEP level information set in ventilator system 112 is transmitted to processor 216 in real-time (or is transmitted to storage 212, for processing at a later time) for processing and output as a graphical representation for display in first graph image area 310 of display screen 300.

Second graph image area 320 is configured for displaying a FiO₂ graph 324 representing the FiO₂ level (as a percentage) set in ventilator system 112 as a function of time, e.g., for the past 7 days (although other timeframes are also contemplated). FiO₂ graph 322 includes an actual FiO₂ indicator line 326 (the broken line) and a daily low FiO₂ indicator line 328 (the solid line) superimposed on one another. The area under the daily low FiO₂ indicator line 328 is shaded or colored in accordance with a coding protocol, which will be described below. Further, alert indicators 329 may be provided in graph 324 at particular points of concern, as will also be described below. As can be appreciated, FiO₂ level information set in ventilator system 112 is transmitted to processor 216 in real-time (or is transmitted to storage 212, for processing at a later time) for processing and output as a graphical representation for display in second graph image area 320 of display screen 300.

Third graph image area 330 is configured for displaying a temperature graph 334 representing the patient's measured temperature (shown in FIG. 3 in ° C.) as a function of time, e.g., for the past 7 days (although other timeframes are also contemplated). Temperature measurements may be taken at pre-determined intervals or continuously, e.g., using temperature sensor 122. Portions of temperature graph 334 are shaded or colored in accordance with a coding protocol, which will be described below. As can be appreciated, temperature measurements received by temperature sensor 122 are transmitted to processor 216 in real-time (or are transmitted to storage 212, for processing at a later time) for processing and output as a graphical representation for display in third graph image area 330 of display screen 300.

Fourth graph image area 340 is configured for displaying a graph 344 representing the patient's WBC count (in WBCs/mcL) as a function of time, e.g., for the past 7 days (although other timeframes are also contemplated). WBC count measurements are taken at pre-determined intervals, e.g., once per day, using standard blood analysis techniques. The WBC count measurement is input into, for example, bedside device 130 or one of remote devices 170, for transmission processor 216 (or storage 212) for processing and output as a graphical representation for display in fourth graph image area 340 of display screen 300. Portions of WBC count graph 344 are shaded or colored in accordance with a coding protocol, which will be described below.

Text boxes 352, 354, 356, as mentioned above, display relevant patient information including biographical information, e.g., the patient's name, age, etc., and hospital record information, e.g., the patient's drug administration schedule, the number of days-on-ventilator, or other relevant information. The information displayed in text boxes 352, 354, 356 of display screen 300 may be input, for example, via bedside device 130, one of remote devices 170, or may be pulled from electronic files or records in other locations via server(s) 140, 150, 160.

Status indication area 360 of display screen 300 is provided for displaying a status indicator 362 as a function of the current and trending PEEP levels, FiO₂ levels, patient temperature, patient WBC count, and/or other information. Status indicator 362 may indicate a patient's risk level of experiencing a VAE, that a VAE has occurred in the past, and/or that a VAE is ongoing. More specifically, depending on the current and/or trending PEEP levels, FiO₂ levels, patient temperature, and patient WBC count, and in accordance with other information, a risk status or current status is determined and displayed in status indication area 360. For example, status indication area 360 may remain blank where the above-noted information indicates that the patient is in stable condition with a relatively low risk of VAE; a medium-risk indicator, e.g., a yellow icon, may be provided where current and trending information indicates that the patient's risk of experiencing a VAE is increasing; and a red icon may be provided where a VAE has occurred, is ongoing, or where the patient is at a high risk for experiencing a VAE. Other indicator systems are also contemplated. Determination of the above-described status for display in status indication area 360 is detailed below.

Referring now to FIG. 4, another display screen configured to display current and trending information relating to PEEP, FiO₂, temperature, and WBC count, respectively, as output by UI 218 (FIG. 2), is shown generally identified by reference numeral 400. Similar to display screen 300 (FIG. 3), display screen 400 may, depending on the configuration of system 10, be viewed one of the patient care devices 110, patient monitoring devices 120, bedside devices 130, remote devices 170, or any other suitable device. Display screens 300 (FIG. 3), 400 may include any or all of the features illustrated in the figures of the other display screen 300 (FIG. 3), 400. Accordingly, only differences between display screens 300 (FIG. 3) and 400 will be described in detail below, while similarities will only be summarily described or omitted entirely. Further, display screen 400 may be configured as an initial screen that leads to display screen 300 (FIG. 3) or portions thereof. That is, display screen 400 may be provided as a summary screen, through which the graphs and other information provided on display screen 300 (FIG. 3) are accessible, e.g., by selecting the appropriate feature on display screen 400. Zoom features, rotation features, swiping features, and other application features known in the art may likewise be incorporated into display screen 300 (FIG. 3) and/or display screen 400, depending on the device displaying the particular display screen 300 (FIG. 3), 400.

Display screen 400 includes first, second, third, and fourth display areas 410, 420, 430, 440 corresponding to PEEP information, FiO₂ information, temperature information, and WBC count information, respectively. Additional patient information is displayed in areas 450, 460 of display screen 400, similarly as described above with respect to display screen 300 (FIG. 3). A status indicator 470 for displaying a status of the patient based on current and/or trending information is also provided.

As opposed to providing graphs representing the PEEP information, FiO₂ information, temperature information, and WBC count information, as in display screen 300 (FIG. 3), display screen 400 displays numerical values 412a, 422a, 432, 442 corresponding to the current (or most-recent) PEEP level, FiO₂ level, temperature, and WBC count, respectively, of the patient, and trending icons 414, 424, 434, 444 corresponding to the overall trend in PEEP level, FiO₂ level, temperature, and WBC count, respectively, of the patient. Numerical values 412b, 422b corresponding to the daily-low PEEP and FiO₂ levels, respectively, may also be provided. Numerical values 412a and 412b, 422a and 422b, 432, 442 may further be outlined, surrounded, backed, or otherwise associated with a color-coding protocol similar to the color-coding of graphs 314, 324, 334, 344 of display screen 300 (FIG. 3), which will be described in greater detail below. Trending icons 414, 424, 434, 444 may include, for example, an up arrow (indicating an increase in the particular value), a down arrow (indicating a decrease in the particular value), or a horizontal bar (indicating no change in the particular value). Trending icons 414, 424, 434, 444 may also be outlined, surrounded, backed, or otherwise associated with a color-coding protocol similar to the color-coding of graphs 314, 324, 334, 344 of display screen 300 (FIG. 3). As mentioned above, display screen 400 may be configured as an initial screen wherein the graphs and other information provided on display screen 300 (FIG. 3) are accessible by selecting the appropriate feature on display screen 400. For example, selecting one of trending icons 414, 424, 434, 444 may display a full-screen graph (see FIG. 3) corresponding to that feature. Alternatively, the graphs may be display on display screen 400 instead of trending icons 414, 424, 434, 444, with the similar full-screen capability upon selecting one of the graphs.

Referring generally to FIGS. 1-4, and as mentioned above, certain parameters, conditions, and/or sensed data, alone or in combination with other parameters, conditions, and/or sensed data, have been found to indicate an increased risk that a patient may suffer a VAE, or that a VAE is occurring, about to occur, or has occurred. In particular, it has been found that the need for sustained increase in the PEEP level set in ventilator system 112, e.g., to account for a patient's increased oxygenation needs, after a stable (or decreasing) period may be caused by a VAE. Likewise, the need for sustained increase in the FiO₂ level set in ventilator system 112, e.g., to account for a patient's increasing oxygenation needs, after a stable (or decreasing) period may also be caused by a VAE. Further, the need to start a patient on a new antimicrobial agent, or drug, e.g., to combat a potential infection, may be caused by a VAE. An increase in patient body temperature and/or WBC count may also indicate a potential infection, illness, or other condition which may be caused by a VAE. Other patient information, parameters, and/or data may additionally or alternatively be used.

With respect to increasing PEEP levels, increasing FiO₂ levels, and administering a new antimicrobial drug, these steps are often taken in response to a particular symptom, condition, or state of the patient. That is, these steps are reactionary. However, the need to increase these levels or administer new drugs, as detailed above, may also be indicative of a VAE that is occurring, about to occur, or has occurred. Thus, by monitoring the occurrences and trends of these reactions, a risk level regarding a patient's susceptibility to suffering a VAE can be determined and observed data during and after a VAE can be obtained. Although changes in body temperature and WBC count are not normally reactionary in terms of actions taken by medical personnel in response to a particular symptom, condition, or state, increase in body temperature and WBC count is often a natural reaction to an underlying issue. Thus, monitoring body temperature and WBC count can also be beneficial in identifying an increased risk of, an on going, or a past underlying issue, e.g., a VAE.

Each of the above-noted indications, e.g., PEEP levels, FiO₂ levels, administration of new drugs, patient temperature, and WBC count, can be conveniently monitored, in real-time or as new data for these indications is received (whether automatically sensed or manually input), by medical personnel in accordance with the present disclosure by viewing either display screen 300 (FIG. 3) or display screen 400 (FIG. 4). Display screens 300, 400 (FIGS. 3 and 4, respectively) provide current, real-time, or most recent information as well as trend information regarding each of these indicators. More specifically, with respect to display screen 300, a user is able to readily ascertain whether PEEP levels or FiO₂ levels have been at a sustained increased level after a stable (or decreasing) period by viewing graphs 314, 324, respectively. For example, by viewing display screen 300, the user can readily ascertain that the PEEP level was increased on day 5 after 4 days of decreasing or steady PEEP levels and that the FiO₂ level was likewise increased on day 5 after 4 days of decreasing or steady FiO₂ levels. Further, although sustained increase would not be determinable on day 5, the necessitated increase in PEEP and FiO₂ levels provide medical personnel with at least an indication that the potential risk of the patient suffering a VAE has increased. Alert indicators 319, 329 may indicate such increased risk points. That is, system 210 (FIG. 2) may be configured to display an alert indicator 319, 329 on display screen 300 to draw attention to the point-in-time where PEEP or FiO₂ levels are increased, as this may be the initial indication of a potential VAE. Text or audible notifications, both local and remote, may also be provided to indicate this potential VAE.

With respect to display screen 400 (FIG. 4), although graphs are not presented initially (though they may be accessible via selecting an appropriate icons) numerical values 412a, 422a, 432, 442 and trending icons 414, 424, 434, 444 provide medical personnel with similar information. That is, with trending icon 414 or 424 indicating an increasing value, the user is alerted to the fact that PEEP or FiO₂ levels, respectively, have been increased, thus indicating a potential VAE. Increasing (or decreasing) temperature and WBC counts can likewise be correlated in real-time with the PEEP or FiO₂ levels and trends thereof.

Once a sustained increase in PEEP or FiO₂ levels is detected, e.g., by day 7, as illustrated in graphs 314, 324, where increased PEEP and FiO₂ levels have been sustained for 2 days, medical personnel can readily ascertain that a serious condition exists, the patient is at a high risk of VAE, or a VAE is ongoing. Text or audible notifications, both local and remote, may also be provided at this point to alert the appropriate personnel. The alerts and/or notifications provided at the various stages, e.g., the notifications on day 5 and day 7, may differ in type, volume, size, length, etc., to indicate a severity or urgency of that particular notification and well as relative to other notifications.

Similarly with respect to temperature and WBC count, as indicated in graphs, 334, 344, respectively, increases can be noted in real-time or as each of these indicators is updated whether automatically sensed (as in, for example, temperature) or manually input (as in, for example, WBC count), as can trending information. In particular, not only can increases in temperature and WBC count be determined, but occurrences of such increases can be seen in temporal relation relative to the changes and trends in PEEP and FiO₂ levels. Such a feature is important in that increased temperature and/or WBC count occurring near, e.g., within two days, increased PEEP or FiO₂ levels is indicative of a VAE and, in particular, an IVAC or VAP.

The above-described display screens 300, 400 (FIGS. 3 and 4, respectively) not only present relevant information regarding the levels or values of each indicator individually and in relative temporal relation to facilitate determinations by medical personnel, but also provide alerts, color-coding, and status indicators which may be at least partially determined by the current values or trending values of one or more of the indicators. System 210 and, in particular processor 216, processes the various data and information received to provide alerts, color-coding, and status indicators taking into consideration the values and trends of one or more of the indicators.

Referring to FIG. 3 in particular, with respect to color-coding on display screen 300, where PEEP or FiO₂ levels are stable (or decreasing), the area under curves 318, 328, respectively, may be shaded with a first color or pattern, e.g., yellow. Upon increase in the PEEP or FiO₂ level after the stable (or decreasing) period, the area under curves 318, 328, respectively, may be shaded with a different, second color or pattern, e.g., red, thus alerting medical personnel that a potentially significant change has occurred. However, it is also contemplated that a plurality of levels of color coding be used to provide more granularities with respect to the alert condition. For example, upon initial increase of PEEP or FiO₂ levels, the area under curves 318, 328, respectively, may be changed to a first color, e.g., yellow, upon further increase to an intermediate color, e.g., orange, followed by a change to the second color, e.g., red, upon sustained increased levels, e.g., after two days of increased levels.

Referring once again to FIG. 4, with respect to color-coding on display screen 400, numerical values 412b, 422b and/or trending icons 414, 424 may likewise be outlined, surrounded, backed, or otherwise associated with a first color, e.g., yellow, where PEEP or FiO₂ levels are stable (or decreasing), a second color, e.g., orange, where PEEP or FiO₂ levels have recently increased, and a third color, e.g., red, where sustained increase of PEEP or FiO₂ levels has occurred.

Turning back to FIG. 3, color-coding of graphs 334 and 344 may accomplished by providing one or more color bands corresponding to different alert levels of temperature and WBC count, respectively. These bands may be determined in accordance with previous patient data, pre-determined settings, or other factors. As can be appreciated, when graphs 334, 344 indicate a temperature or WBC count, respectively, in one of the color bands, the user is alerted to this potentially significant event. On the other hand, minor variations in temperature and WBC count below these color bands can be given less attention. As an alternative to, or in addition to bands, the graph lines themselves may be changed to exhibit a color or feature corresponding to the particular alert level.

With reference to FIGS. 3 and 4, status indicators 362, 470 may provide similar functionality as the color-coding described above with the additional feature of considering multiple factors in determining a level of alert to display or otherwise provide. That is, a first level indicator may be provided where only one indicator value has been increased, a second level indicator may be provided where two indicator values have been increased, etc. Thus, while one increased value or a single sustained increase may not necessarily indicate a VAE, depending on the circumstances and other considerations, multiple increased values or sustained increased values are more likely to indicate a VAE. Multiple increased values or sustained increased values may also be utilized to determine more information about the risk, severity and/or particular type of VAE.

Alerts and notifications, which may be provided in any suitable form, e.g., as a visual display, audible alert, data save, print-out, message (text, email, voicemail, etc.), or other suitable alert or notification, may be set up for any or all of the above-described features. For example, where a color change is triggered, a color band is entered, an alert indicator 319, 329 is displayed, and/or a status indicator 362, 470 is changed, an alert or notification may be provided.

In addition to alerts and notifications, reports may be automatically generated by processor 216 (FIG. 2) and transmitted or printed in response to fulfillment of particular conditions, depending on protocol or user-settings. These automatically generated repots may include, for example, reproductions of any or all of graphs 314, 324, 334, 344, or may extract data from graphs 314, 324, 334, 344 to create a text-based report summarizing the data. Other information received, generated, and/or stored by system 10 (FIG. 1) may also be utilized in the reports. The reports can then be used to fulfill reporting requirements, e.g., to report occurrences of VAE to the CDC or other reporting agency, for studies, or other internal (or external) purposes without the need for medical personnel to manually fill out the required paper work. For example, reports may be generated and transmitted to an appropriate database for inclusion in the patient's Electronic Medical Record (EMR) and/or an Admission, Discharge, and Transfer (ADT) electronic file. Reports may likewise be generated for collecting lab data for use in a particular laboratory study or studies. As an alternative to or in addition to automatically-generated reports, report requests may be generated in accordance with one or more of the alerts or notifications, thus providing medical personnel with the option of generating a report upon receipt of the particular alert or notification. Further still, manual report requests may be initiated for generating a report for a particular purposes and./or at a particular time, as needed.

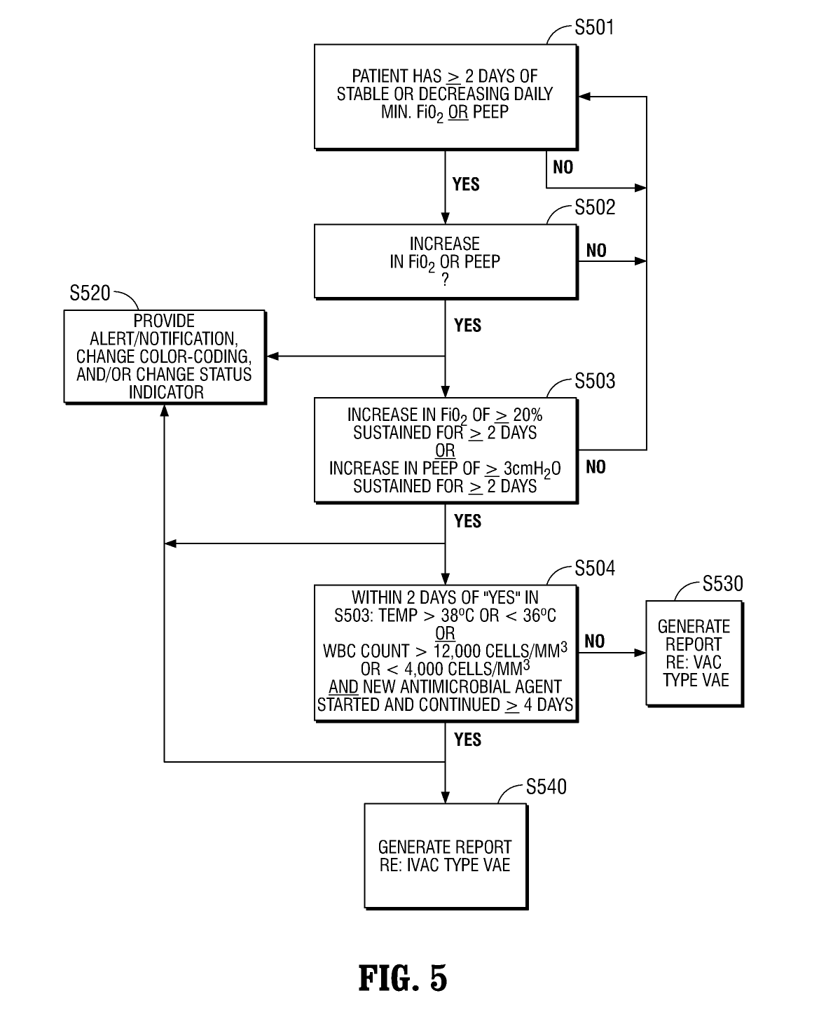

An exemplary implementation of the present disclosure in accordance with the Centers for Disease Control and Prevention's (CDC) VAE protocol for VAE determination is described with reference to FIG. 5, keeping in mind that the present disclosure is equally applicable for use in accordance with any suitable protocols, rules, thresholds, or conditions. As can be appreciated in view of the following, the systems and methods of the present disclosure are fully compatible with and facilitate determination of the occurrence of a VAE, as defined by the CDC. However, in addition to facilitating the determination of the occurrence of a VAE using the particular elapsed time and value thresholds provided by the CDC protocol, the present disclosure provides real-time monitoring, analysis, and alerts and notifications, thus allowing for early identification of a potential VAE or increased risk for a VAE. Other features will also become more apparent in view of the exemplary implementation described below.

Continuing with reference to FIG. 5, initially, as indicated in S501, it is determined whether the patient has had at least two days of stable or decreasing daily minimum FiO₂ or PEEP values. If “NO” in S501, the process loops back to the beginning where S501 is repeated until this condition is satisfied. If “YES” in S501, the process proceeds to S502, where it is determined if there has been an increase in FiO₂ or PEEP. If “NO” in S502, the process loops back to S501. If “YES” in S502, S520 is carried out, wherein system 210 (FIG. 2) provides for, as detailed above, a color-change on the relevant portions of displays 300, 400 (FIGS. 3 and 4, respectively), a change of status indicators 362, 470 (FIGS. 3 and 4, respectively), issuance of an alert or notification, and/or any of the other features detailed above.

In addition, if “YES” in S502, the process proceeds to S503. In S503, it is determined whether there has been an increase in FiO₂ percentage by at least 20 (either an absolute increase of 20% in FiO₂, or a 20% relative increase in FiO₂) that has been sustained for at least two days, or whether there has been an increase in PEEP of at least 3 cmH₂O sustained for at least two days. If “NO” in S503, the process loops back to S501. If “YES” in S503, S520 is carried out similarly as described above and the process proceeds to S504.

In S504, if, within (+/−) two days of the satisfying condition in S503, (1) the patient's temperature is above 38° C. or below 36° C. or the patients WBC count is above 12,000 cells/mm³ or below 4,000 cells/mm³; and (2) a new antimicrobial agent has been started and continued for at least four days, e.g., “YES” in S504, S520 is carried out similarly as described above. In addition, if “YES” in S504, S540 is also carried out, wherein a report is generated including any or all of the information detailed above and indicating the occurrence of an IVAC type VAE in accordance with CDC protocol. If “NO” in S504, S530 is carried out, wherein a report is generated including any or all of the information detailed above and indicating the occurrence of a VAC type VAE in accordance with CDC protocol.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A system for monitoring a patient on ventilation, comprising:
a ventilator system configured to provide mechanical ventilation in accordance with a FiO₂ setting and a PEEP setting;
a temperature sensor configured to sense body temperature;
at least one input device configured to receive patient information, the patient information including WBC count information; and
an information processing system communicatively coupled to the ventilator system, temperature sensor, and input device, the information processing system configured to receive, process, and output FiO₂ setting information and PEEP setting information from the ventilator system, body temperature information from the temperature sensor, and WBC count information from the input device; and
a display device configured to display the FiO₂ setting information, PEEP setting information, body temperature information, and WBC count information output from the information processing system graphically as a function of time.

2. The system according to claim 1, wherein the information processing system is further configured to output at least one of an alert and a notification in response to at least one of a change in the FiO₂ setting information over time and a change in the PEEP setting information over time.

3. The system according to claim 1, wherein the information processing system is further configured to generate a report in response to at least one of a change in the FiO₂ setting information over time and a change in the PEEP setting information over time.

4. The system according to claim 1, wherein the information processing system is further configured to transmit data to or receive data from at least one of a reporting agency, an Electronic Medical Record (EMR) system, an Admission, Discharge, and Transfer (ADT) system, and a system storing lab data.

5. The system according to claim 1, wherein the display device is configured to provide color-coding associated with the graphical display of the FiO₂ setting information and the graphical display of the PEEP setting information.

6. The system according to claim 1, wherein the display device is configured to provide color-coding associated with the graphical display of the body temperature information and the graphical display of the WBC count information.

7. The system according to claim 1, wherein the display device is further configured to display at least one alert indicator indicative of a potential VAE on at least one of the graphical display of the FiO₂ setting information and the graphical display of the PEEP setting information.

8. A method of monitoring a patient on a ventilator, comprising the steps of:
obtaining real-time data indicative of a FiO₂ setting of the ventilator;
obtaining real-time data indicative of a PEEP setting of the ventilator;
displaying data indicative of the FiO₂ setting graphically as a function of time;
displaying data indicative of the PEEP setting graphically as a function of time; and
determining at least one point-of-interest using at least one of the graphical display of data indicative of the FiO₂ setting and the graphical display of data indicative of the PEEP setting.

9. The method according to claim 8, further comprising the steps of:
obtaining real-time data indicative of body temperature; and
displaying real-time data indicative of the body temperature graphically as a function of time, wherein the at least one point-of-interest is determined in accordance with the graphical display of the data indicative of the body temperature.

10. The method according to claim 8, further comprising the steps of:
obtaining up-to-date data indicative of a WBC count; and
displaying data indicative of the WBC count graphically as a function of time, wherein the at least one point-of-interest is determined in accordance with the graphical display of the data indicative of the WBC count.

11. The method according to claim 8, further comprising the step of providing at least one of an alert and a notification upon determination of the at least one point-of-interest.

12. The method according to claim 8, further comprising the step of changing at least one of a status indicator and a color-coding scheme upon determination of the at least one point-of-interest.

13. The method according to claim 8, further comprising the step of generating a report upon determination of the at least one point-of-interest.

14. The method according to claim 8, wherein the at least one point-of-interest is determined in accordance with an increase in at least one of the FiO₂ setting and the PEEP setting reflected in the respective graphical displays.

15. The method according to claim 8, wherein the step of determining at least one point-of-interest further includes determining a potential VAE.

16. The method according to claim 8, further comprising the steps of:
obtaining drug administration information; and
displaying the drug administration information.

17. A non-transitory computer-readable storage medium encoded with a program that, when executed by a processor, causes the processor to perform the steps of:
obtaining data indicative of a FiO₂ setting of the ventilator;
obtaining data indicative of a PEEP setting of the ventilator;
displaying the data indicative of the FiO₂ setting graphically as a function of time;
displaying the data indicative of the PEEP setting graphically as a function of time; and
providing an indication of at least one point-of-interest on at least one of the graphical display of data indicative of the FiO₂ setting and the graphical display of data indicative of the PEEP setting.

18. The medium according to claim 17, wherein the step of providing the indication further includes outputting at least one of an alert and a notification regarding the at least one point-of-interest.

19. The medium according to claim 17, wherein the step of providing the indication includes at least one of a displayed status indicator and a displayed color-coding.

20. The medium according to claim 17, wherein the step of providing the indication further includes generating a report regarding the at least one point-of-interest.
